(19) United States
(12) Patent Application Publication
(10) Pub. No.: US 2021/0137727 A1
(43) Pub. Date: May 13, 2021

(54) POROUS OXYGEN ACTIVATED HEATER CONFIGURATIONS, DESIGNS AND APPLICATIONS

(71) Applicant: RECHARGEABLE BATTERY CORPORATION, College Station, TX (US)

(72) Inventors: Adam Laubach, College Station, TX (US); Ross A. Lee, Chesapeake City, MD (US); Darko Marquez, College Station, TX (US); Charles Sesock, College Station, TX (US)

(21) Appl. No.: 16/635,767

(22) PCT Filed: Aug. 1, 2018
(86) PCT No.: PCT/US2018/044883

§ 371 Date: Jan. 31, 2020

Related U.S. Application Data
(60) Provisional application No. 62/539,982, filed on Aug. 1, 2017.

(51) Int. Cl. A61F 7/03

(52) U.S. Cl. CPC A61F 7/032; A61F 2007/0003

(57) ABSTRACT

A device for the delivery of aromas having a package having a front panel and a back panel, a heater disposed inside the package between the front panel and the back panel, and an air delivered composition, the air delivered composition being positioned to receive heat from the heater when the heater is activated, wherein the air delivered composition is volatilized by the heater when activated causing the air delivered composition be delivered through the air.

### CROSS REFERENCE TO RELATED APPLICATIONS

This application claims priority to U.S. Provisional Application Ser. No. 62/539,982 filed on Aug. 1, 2017, the contents of which are fully incorporated herein by reference.

### FIELD OF THE INVENTION

The invention relates to a heater that uses oxygen as a fuel source for a reaction that produces heat, and more specifically to various heater additions/elements/configurations which have improved/extended/controlled heat release, and when combined with packaging and target thermal mass improvements have overall system improved thermal transfer and conservation. The invention also relates to a delivery device for delivering a deliverable such as an aroma, scent, insecticide or repellant through the air.

### BACKGROUND OF THE INVENTION

Air activated heaters are currently used in a variety of applications, for example heating comestible or cosmetic and personal care items. These heaters may include a heater sheet or substrate which includes a reducing agent which provides an exothermic reaction when exposed to oxygen, with the substrate being packaged between a top and bottom film layer. These heaters also typically include an electrolyte for triggering the reaction once the reducing agent is exposed to oxygen.

Known heaters may also include an air access or air diffuser layer which has one or more apertures through which air and oxygen from the atmosphere may pass to reach the heater substrate. The air access or air diffuser layer may be formed as part of the top or bottom film layer or may be a separate layer positioned adjacent to the top of the bottom film layer. The apertures formed in the air access or air diffuser layer may be selectively blocked and unblocked using a removable and/or re-attachable label or flap. When the label, flap or other sealing body is removed, the apertures may be exposed to the atmosphere, with air (and consequently oxygen) passing through the air access or air diffuser layer reaching the heater sheet or substrate, causing the exothermic reaction to begin. Utilizing an air diffuser layer, for example, provides for better reaction control as the diffuser layer can control how much ambient air, and consequently oxygen, reaches a heater substrate within the heater.

### SUMMARY OF THE INVENTION

The present invention is directed to is directed to devices for delivering aromas and heaters which have improved, extended, better controlled heat release, have improved thermal transfer and conservation within the heater. The heaters may also have improved shelf-life and have more efficient heat generation/production and incorporate elements which are triggered when the heater is activated. It should be understood that the though the various embodiments may at times be discussed separately herein, any combination of improvements, layers or heater or package elements may be incorporated into a single heater or air deliverable devices incorporating heaters unless otherwise indicated.

According to one aspect of the invention, a device or heater for the delivery of a deliverable by air is provided. The device includes a package having a front panel and a back panel and a heater disposed inside the package, between the front panel and the back panel. The device further includes a volatile composition/an air delivered composition which delivers an aroma, scent, fragrance, repellant, insecticide and other compounds through the air. The air delivered composition is positioned to receive heat from the heater when the heater is activated, wherein the air delivered composition is volatilized by the heater when activated causing the air delivered composition to release or provide the air delivered composition contained therein through the air. The air deliverable composition may be reactive or releasable by heat, may be, for example, an aromatic, a scent, an insecticide or a repellant. Though discussed as a device for the delivery of a deliverable by air having a heater integrated therein, it should be understood that the device itself may be formed as a heater having an air delivered composition integrated therewith. It should be understood that the air delivered composition may be integrated with any heater discussed herein, including in combination with any elements or layers disclosed in the various heaters discussed herein. By the same token, any combination of heater elements or layers discussed herein may be combinable with any delivery device contemplated by the invention.

In its most basic state, the heater includes a heater element, such as a heater substrate or heater sheet, which has a reducing agent which generates heat in the presence of oxygen. The heater further includes an electrolyte solution, the electrolyte solution being an activator which causes the reducing agent to activate and generate heat when exposed to oxygen. The electrolyte solution may be integrated with the heater element, like for example absorbed into a heater substrate or heater sheet, or alternatively may be absorbed into wicking layer formed as a pad or other absorbent carrier which is placed adjacent the heater element within the package. The heater element may be configured and an amount of electrolyte applied to result in a heater element which has a wet porosity in the range of 10%-90%, and more preferably in the range of 35%-80%, and more preferably yet in the range of 50%-80%.

The heater and/or package may also include additional materials or layers incorporated therein. For example, a moisture absorber may be sealed within the package. The heater element itself may comprise the moisture absorber in addition to the reducing agent and any binders, fillers or other material. Alternatively, the moisture absorber may be formed as an additional substrate within the package. The package may also include a heat retention material which is placed in thermal contact with the heater element or formed as part of the heater element along with the reducing agent and any binders, fillers or moisture absorbing elements included therein. Alternatively, or additionally, a heat retention material may also be integrated with one of the first or second panels forming the package. The package may also, or alternatively, include a separate compart within or isolated from the thermal element which is in thermal contact with the heater element and houses the heat retention material. The heat retention material may be a phase change material which undergoes a phase change from a solid to a liquid when heat is applied to the material, the phase change material controlling a temperature emitted by the device while undergoing the phase change. The phase change material may continue to emit heat substantially at the melting temperature when the substrate is deactivated and the phase change material undergoes a second phase change from the liquid back to the solid. The package may also include at least one reflective layer, the at least one reflective layer being positioned adjacent either one or more of the front panel or the back panel. The at least one reflective layer may be a metalized film, like for example a foil film, and may be formed as a separate layer inside the package or printed on an interior side of one or both of the front panel and the back panel. The package may also include an insulation layer positioned adjacent one or both of the front panel or the back panel. The delivery device or heater may also include a porous insulator having an opening for receiving the package, the porous insulator being oxygen porous to allow oxygen to reach an interior of the porous insulator and the package contained therein. The air delivered composition, or an additional air delivered composition may be integrated with the porous insulator. The porous insulator may also be waterproof.

The air delivered composition may be incorporated into the device in any number of ways. For example, the air delivered composition may be integrated with the heater element as an oil or other liquid element—like for example an aromatic oil—which is absorbed by the heater element, or as a solid element which forms a portion of the heater element along with the reducing agent and any binders, fillers or other aforementioned elements incorporated into the heater element. The air delivered composition may also be integrated with the electrolyte solution in a wicking layer formed by a pad or other absorbent material. The device may include a second pad separate and apart from any wicking layer containing any electrolyte solution, wherein the air delivered composition is integrated with second the pad, the second pad being disposed inside the package so as to receive heat from the heater when the heating element is exposed to oxygen and activated. Any wicking layer or pad may be disposed adjacent the heating element.

In some embodiments of the invention, the front and back panels forming the package may both be air impermeable, wherein at least one of the front panel or the back panel includes at least one air access opening. The package may further include at least one removable seal affixed to at least one of the front panel or the back panel covering the at least one air access opening. The at least one removable seal may be resealable over the at least one air access opening. In addition, the package may further include at least one air diffuser, the at least one air diffuser being disposed inside the package and positioned between the at least one air access opening and the heater element. At least a portion of the air delivered composition, and/or when used in the package any heat retention element, may be integrated with the at least one air diffuser. The at least one air diffuser may be waterproof or formed as a waterproof layer.

In some embodiments of the invention where the front and back panels are air impermeable the package may be divided into at least two cavities wherein the heater element may be positioned in a first cavity or area within the package and the air delivered composition may be positioned in a second cavity area within the package, with the first and second cavities or areas being formed as a portion of the package. In such embodiments, the package is flexible so that the heater element and the scent agent which are separated within the package can be brought in thermal contact with one another as desired. For example, the package may be foldable along an axis or dimension of the package so that the first cavity or area can be brought into thermal contact with a second cavity or area, and heat generated by the heating element when activated can be thermally transmitted through the first cavity or area and the front panel or the back panel of the package and the second cavity or area of the housing to activate the air delivered composition.

In some embodiments of the invention, the front panel and the back panel of the package may both be air permeable. In embodiments where the front and back panel are both air permeable, the device may further include a sachet having a first panel and a second panel, both the first panel and the second panel being formed from a material which is air impermeable with the package being sealed therein. In order to access the package and/or activate the device, the sachet includes one or more removable portions, wherein removal of the one or more removable portions exposes the package to air and consequently oxygen to activate the heater element and the device. The sachet maybe resealable after the one or more removable portions are removed. When the front and back panel of the package are air permeable, one or more of the front panel or the back panel may be waterproof. The package may be removably disposed in the sachet or may be fixed in place therein.

Regardless of whether the package is formed from air permeable or air impermeable panels, rather than be positioned within a single cavity with the heater element, the air delivered composition may be positioned in a second cavity integrated with the package. As explained above, the scented element may be integrated in a second cavity within the package, or alternatively, the second cavity may be attached to an exterior of one of the front panel or the back panel of the package using adhesive or the like.

The device may further include a substrate or carrier substrate which may have multiple elements of the heater and device integrated therewith. For example, one or more of the heater element, the electrolyte or the air delivered composition may be printed on the substrate. Additional elements which may be integrated with the heater may be printed on the substrate as well, such as any moisture absorbing materials or any heat retention materials.

The device may be designed as an insert which can be integrated or inserted into a garment or a patch which may be worn on the body. The device may also include an applicator, the applicator being integrated with an exterior surface of one of the first panel or second panel of the package.

According to one aspect of the invention, a heater or heating device is provided. The heating device includes a housing or package having a first film layer and a second film layer. The heater includes a heating element which includes a reducing agent which generates heat in the presence of oxygen. The heating element may be a heater substrate or heater sheet, for example. The heater further includes an electrolyte solution, the electrolyte solution being an activator which causes the reducing agent to activate and generate heat when exposed to oxygen. The substrate and the electrolyte solution are housed within the package or housing and are sealed between the first film layer and the second film layer. At least one removable portion is provided which can be removed to allow air from outside the housing or package to reach an interior of the housing or package and the substrate and the reducing agent therein.

The heater element, including any electrolyte added thereto may have a wet porosity in the range of 10%-90%, and more preferably in the range of 35%-80%, and more preferably yet in the range of 50%-80%, after the electrolyte solution is directly or indirectly added to the heater element.

The heater may also include any combination of additional materials or layers incorporated therein.

For example, a moisture absorber may be sealed within the housing or package. The heater element itself may comprise the moisture absorber in addition to the reducing agent and any binders, fillers or other materials. Alternatively, the moisture absorber may be formed as an additional substrate within the housing or package.

The heater may also, or alternatively, include a heat retention material which is placed in thermal contact with the heater element or formed as part of the heater element along with the reducing agent and any binders, fillers or moisture absorbing elements included therein. Alternatively, or additionally, a heat retention material may also be integrated with one of the first or second film layers forming the housing or package. The heater may also, or alternatively, include a separate compartment within or isolated from the heating element which is in thermal contact with the heater element and houses the heat retention material. The heat retention material may be a phase change material which undergoes a phase change from a solid to a liquid when heat is applied to the material, the phase change material controlling a temperature emitted by the device while undergoing the phase change and continuing to emit heat substantially at the melting temperature when the substrate is deactivated and the phase change material undergoes a second phase change from the liquid back to the solid.

The heater may also include at least one reflective layer, the at least one reflective layer being positioned adjacent either one or more of the first or second film layers of the housing or package. The at least one reflective layer may be a metalized film, like for example a foil film, and may be formed as a separate layer inside the housing or package or printed on an interior side of one or both of the first film layer or the second film layer.

The heater may also include an insulation layer positioned adjacent one or both of the first film layer or the second film layer.

The heater may also include a porous insulator having an opening for receiving the heater, the porous insulator being oxygen porous to allow oxygen to reach an interior of the porous insulator and the heater contained therein. An air delivered composition may be integrated with the porous insulator. The porous insulator may also be waterproof.

According to one aspect of the invention, an application device is provided. The application device includes a substrate forming an application medium. A reducing agent is integrated with the substrate, the reducing agent generating heat when activated and exposed to oxygen. An electrolyte solution is also integrated with the substrate, the electrolyte solution being an activator which causes the reducing agent to activate and generate heat when exposed to oxygen. An aromatic agent may also be integrated with the substrate, the aromatic agent generating an aroma when heated by the reducing agent when the reducing agent is exposed to oxygen and activated to generate heat.

The substrate may be liquid or semi-solid at room temperature, like for example a cream, lotion or cosmetic compound. The substrate may also be a solid at room temperature and formed as, for example, a pad, sponge, non-woven or brush to apply a make-up compound.

According to one aspect of the invention, a heating device having a housing formed from a first film layer and a second film layer is provided. The heater includes a heating element, having a reducing agent which generates heat in the presence of oxygen and an electrolyte solution, the electrolyte solution being an activator which causes the reducing agent to activate and generate heat when exposed to oxygen. The heating element and the electrolyte solution are housed within the housing and sealed between the first film layer and the second film layer. At least one removable portion which can be removed to allow oxygen from outside the housing to reach an interior of the housing and the heating element and the reducing agent therein. The heater further includes an applicator, the applicator being integrated with an exterior portion of the housing.

Other advantages and aspects of the present invention will become apparent upon reading the following description of the drawings and detailed description of the invention.

### BRIEF DESCRIPTION OF THE DRAWINGS

The present invention will become more fully apparent from the following description and appended claims, taken in conjunction with the accompanying drawings. Understanding that the accompanying drawings depicts only typical embodiments, and is, therefore, not to be considered to be limiting of the scope of the present disclosure, the embodiments will be described and explained with specificity and detail in reference to the accompanying drawings as provided below.

FIG. 1 shows an embodiment of an aroma delivery device as contemplated by the invention;

FIG. 2 shows an exploded view of the embodiment shown in FIG. 1;

FIG. 3 shows an embodiment of an aroma delivery device as contemplated by the invention;

FIG. 4 shows an exploded view of the embodiment shown in FIG. 3;

FIG. 5 shows an embodiment of an aroma delivery device as contemplated by the invention;

FIG. 6 shows an exploded view of the embodiment shown in FIG. 5;

FIG. 7 shows an embodiment of an aroma delivery device as contemplated by the invention;

FIG. 8 shows an exploded view of the embodiment shown in FIG. 7;

FIG. 9 shows an embodiment of an aroma delivery device as contemplated by the invention;

FIG. 10 shows an exploded view of the embodiment shown in FIG. 9;

FIG. 11 shows the embodiment of FIG. 7 being folded about a dimension of the device;

FIG. 12 shows the embodiment of FIG. 9 being folded about a dimension of the device;

FIG. 13 shows an embodiment of an aroma delivery device as contemplated by the invention;

FIG. 14 shows an exploded view of the embodiment shown in FIG. 13;

FIG. 15 shows an embodiment of an aroma delivery device as contemplated by the invention;

FIG. 16 shows an exploded view of the package 12′ of the embodiment shown in FIG. 15;

FIG. 17 shows the embodiment of FIG. 15 with the removable portion 46 removed and package 12′ being removed from sachet 40;

FIG. 18 shows an embodiment of a garment integrated with a device 10 as contemplated by the invention;

FIG. 19 shows an embodiment of a garment integrated with a device 10 as contemplated by the invention;

FIG. 20 shows an embodiment of a heater as contemplated by the invention;

FIG. 21 shows an exploded view of the embodiment shown in FIG. 20;

FIG. 22 shows a graphical representation of the reaction properties of a heater element contemplated in any embodiment of the invention;

FIG. 23 shows a heater element as contemplated by an embodiment of the invention;

FIG. 24 shows an embodiment of a heater as contemplated by the invention;

FIG. 25 shows an exploded view of the embodiment shown in FIG. 24;

FIG. 26 shows an embodiment of a heater as contemplated by the invention;

FIG. 27 shows an exploded view of the embodiment shown in FIG. 26;

FIG. 28 shows an embodiment of a heater as contemplated by the invention;

FIG. 29 shows an embodiment of a heater as contemplated by the invention;

FIG. 30 shows an exploded view of the embodiment shown in FIG. 29;

FIG. 31 shows an embodiment of an applicator as contemplated by the invention; and

FIG. 32 shows an embodiment of an applicator as contemplated by the invention.

### DETAILED DESCRIPTION OF THE INVENTION

While this invention is susceptible of embodiment in many different forms, there is shown in the drawings and will herein be described in detail one or more embodiments with the understanding that the present disclosure is to be considered as an exemplification of the principles of the invention and is not intended to limit the invention to the embodiments illustrated.

Reference throughout this description to features, advantages, objects or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, any discussion of the features and advantages, and similar language, throughout this specification may, but does not necessarily, refer to the same embodiment, and advantages described in different devices, heaters or embodiments are combination to achieve the present invention.

FIGS. 1-17 show various delivery device 10 for delivering a deliverable through the air as contemplated by the invention. As shown in FIGS. 1-17, each device, which may also be formed as a heater, includes a package 12 which includes a front panel 14 and a back panel 16 surrounding an interior 18. A heater 20 is disposed within interior 18 between the front panel 14 and the back panel 16. Each device further includes a volatile composition or an air delivered composition incorporated into the device. The air delivered composition is positioned either within the package or integrated with the package so that the air delivered composition is in thermal contact with the heater upon heater activation so that the air delivered composition is volatized or activated upon activation of the heater. Heating of the air delivered composition causes delivery of the air delivered composition through the air. The air delivered composition may be an aroma, scent, fragrance, insecticide, repellant or other composition or compound.

In each embodiment shown in FIGS. 1-17, heater 20 may be formed and include layers as are known in the art. For example, heater 20 may include a heating element 22 which may be a heater sheet or heater substrate which includes a reducing agent which generates heat in the presence of oxygen. Reducing agents may be any reducing agents known in the art. In addition to the reducing agent, as is known in the art the heater element may include further elements like fillers or binding agents in order to help hold together or maintain a heater sheet or heater substrate. The heater element may also include a carrier substrate to which the reducing agent is applied or integrated with, like for example a pad.

The heater 20 further includes an electrolyte solution which is an activator which causes the reducing agent to generate heat when the reducing agent is exposed to oxygen. The electrolyte solution may be any electrolyte solution known in the art.

The electrolyte solution may be incorporated into the heater 20 in various manners. For example, the electrolyte solution may be incorporated directly into the heater element in a device, by for example, applying the electrolyte solution to heating element 22 in FIGS. 1 and 2, with FIG. 2 being an exploded view of the device shown in FIG. 1. Alternatively, as seen in FIGS. 3 and 4, with FIG. 4 being an exploded view of the device shown in FIG. 3, the electrolyte solution may be provided by applying it to a wicking layer 24 which may be a pad or other absorbent material which is placed adjacent the heating element within the package 12 so that the electrolyte solution can be transferred to the heating element from the wicking layer. Utilizing a wicking layer adjacent the heater element allows for manufacture of the heater in oxygen rich environments as the wicking layer may have electrolyte applied to only one side immediately prior to positioning the non-saturated side adjacent the heater sheet or substrate. The front and back panels, particularly when the front and back panels are air impermeable, may then be immediately sealed in an air-tight manner around both the wicking layer and the heating element after the wicking layer is placed adjacent the heater element to cut off oxygen and prevent premature activation of the heater. If done quickly enough, the electrolyte will not reach the opposite side of the wicking layer, and consequently the heater sheet or heater substrate before the front and back panels are sealed around the interior. Though certain embodiments shown herein do not show a wicking layer 24, it should be understood that such a wicking layer may be incorporated into any of the heaters and packages discussed herein.

Rather than use an electrolyte solution, the heater may include an electrolytic salt or the like which may be incorporated or integrated with the heater element. In such embodiments a wicking layer may be provided with water or some other solution which, when combined with the electrolytic salt will form an electrolytic solution which will activate the heater element. Electrolytic salts may be any salts which can be used to generate an electrolyte solution when combined with water or other solution.

The air delivered composition may be a liquid element, like an oil, or a solid element, which is activated emitted or delivered when the air delivered composition is activated by the heating element 22. Depending on the type of air delivered composition used, for example an oil or liquid versus a solid, the air delivered composition may be incorporated into the device 10 and/or heater in any number of ways.

The air delivered composition may be integrated with heater element 22 in FIGS. 1 and 2, for example. Integration may be accomplished by applying or absorbing a liquid or oil, for example, into a heater sheet or heater substrate during manufacture. When a solid air delivered composition is used, the solid may be incorporated directly into the formation of the heater sheet or substrate when the heater sheet or substrate is formed. As a further alternative, one or both of the heater element 22 or the air delivered composition may be printed onto a single carrier substrate, resulting in a device similar to those shown in FIGS. 1-4. In such embodiments, the reducing agent of the heater element may be printed on a first side of a carrier substrate, while an air delivered composition is printed on a second side of the carrier substrate or absorbed by the carrier substrate. The electrolyte solution may also be absorbed by the carrier substrate or integrated with the heater element and reducing agent during manufacture. Alternatively, the electrolyte solution may be integrated with a wicking layer 24 which is positioned adjacent and in contact with the heater element.

Alternatively, the air delivered composition may be integrated with the electrolyte solution and any wicking layer 24 used to carry the electrolyte solution as seen in FIGS. 3 and 4. Rather than be incorporated into a single wicking layer with the electrolyte solution, the air delivered composition may be absorbed into a separate wicking layer or pad 26 as seen in FIGS. 5 and 6 with FIG. 6 being an exploded view of the device shown in FIG. 5. Such packages may include multiple wicking layers or pads 24, 26 as seen in FIGS. 5 and 6, or a single wicking layer or pad 26 containing only the scented element as seen in FIGS. 7 and 8 with FIG. 8 being an exploded view of the device shown in FIG. 7. When a single pad 24 is used to provide both the electrolyte solution and the air delivered composition as shown in FIGS. 3 and 4, the pad 24 should be placed adjacent the heater element to ensure that the electrolyte solution reaches the heater element 22 to activate the heater element 22 once exposed to oxygen. Where a separate wicking layer or pad 26 is used to carry the air delivered composition, or any further elements discussed herein other than the electrolyte solution, the separate wicking layer or pad 26 may be placed adjacent the heater element 22, but only need to be placed in a position where the separate wicking layer or pad 26 is in thermal contact, or can be placed in contact with, the heater element 22 in order to activate the air delivered composition when the heater element 22 and reducing agent are activated and exposed to oxygen.

As a further alternative, as seen in FIGS. 9 and 10, with FIG. 10 being an exploded view of the device shown in FIG. 9, the air delivered composition may be formed as a separate substrate 27. Similar to a separate wicking layer or pad, substrate 27 may be positioned adjacent heater element 22, however need only be positioned within device 10 in a manner where substrate 27 is in thermal contact, or can be placed in thermal contact, with the heater element when the heater element is activated in order to activate the air delivered composition and release aromas therefrom.

The front panel 14 and back panel 16 of the package 12 and device may be formed using either air permeable or air impermeable materials.

In the embodiments shown in FIGS. 1-14, for example, front panel 14 and back panel 16 may be formed using air impermeable films which can be sealed in an air-tight manner around the heater 20 and the air delivered composition so that activation of the heater can be prevented by package 12 without using any external housing, packaging or materials. In order to allow air and consequently oxygen to reach the heater element to begin the heat generating reaction with the reducing agent, as best seen in FIGS. 2, 4, 6, 8, 10 and 14, one or both of the front panel or back panel may include air access openings 28 which allow air, and consequently oxygen, from the atmosphere outside package 12 to reach interior 18 and heater 20 contained therein.

In order to prevent access of air and consequently oxygen from reaching interior 18 of package 12 before activation of the reducing agent and air delivered composition is desired, removable seal 30 may be provided to cover and seal air access openings 28. The removable seal may be adhered to the front or back panel over the air access openings in a replaceable manner so that if deactivation of the device is desired before the heater is expired, air and oxygen can be cut off from interior 18 and the reaction stopped.

In order to control the rate air and oxygen reach interior 18 and the reducing agent contained therein, as seen in FIGS. 1-14 air diffuser 32 may be provided within package 12, the air diffuser being positioned between air access openings 28 and heating element 22.

In addition to controlling the rate of oxygen which passes to interior 18 of package 12, air diffuser 32 may also act as a carrier for the air delivered composition, wherein the air delivered composition is integrated with the air diffuser. Integration may occur by absorbing the air delivered composition into the air diffuser or by applying a coating to a portion or all of the air diffuser during manufacture. In addition to carrying the air delivered composition, as discussed herein air diffuser 32 may be used to carry additional elements which may be incorporated into package 10.

In order to prevent moisture from outside package 12 from reaching interior 18 when removable seal 30 is removed, air diffuser 32 may also be a waterproof layer or constructed using waterproof or hydrophobic materials. By making the air diffuser a waterproof layer, device 10 can be used in wet environments while minimizing the possibility that the electrolyte solution and/or reducing agent will be affected by water entering the heater. The waterproofing layer may prevent, for example dilution, washing or erosion of the electrolyte from the wicking layer or of the reducing agent from the heater element. A waterproof air diffuser may also prevent unwanted moisture from entering the heater causing the heater to heat to an undesirable temperature and may prevent water or unwanted moisture from occluding the microstructures within the substrate and the heater as a whole which may prevent oxygen from reaching the reducing agent. A waterproof air diffuser may provide better and more efficient heater usage and transport in environments where existing exothermic heaters cannot be used, for example, in a shower or bathtub and/or in inclement weather or transporting through inclement weather.

In order to make air diffuser 32 both a waterproof layer and an air diffuser, the air diffuser may be formed using a microporous, microperfed, nonwoven, or monolithic film. For example, microporous films which utilize CaCO3 to create the micropores in the films may be utilized for the air diffuser. Films which are needle perfed, laser perfed or femto laser perfed may also be used. Monolithic films such as polyurethane polyether films, polyester polyether films may be used also be used as a waterproof air diffuser.

The selection of material for a waterproof air diffuser layer may be selected based on the degree of moisture resistance desired, the rate of air diffusion which is desired and/or other properties which can be controlled by air diffuser. For example, the material can be selected based upon the ability of the air diffuser to control transmission of air to the heater element via selective air permeation rates that can range from 200 to 1600 air flow cc/cm2/min at 90 psi; 8 to 850 Gurleys (sec for 100 cc of compressed air to permeate 1 in2) and up to over 15,000 oxygen transmission rate (cc/m2/day/atm). When maximum heating or heat rate is required from a device, an air diffuser may be selected in order based on a high air permeation rate. For heaters where a slower reaction is desired, a slower rate material can be selected. The individual material for each an air diffuser can also be manipulated to match a particular air transmission rate, for example by adding additional micropores or perforations in the diffuser.

In addition to a waterproof air diffuser 32 being used to prevent moisture from entering the interior 18 of package 12, a waterproof air diffuser may also be utilized to keep moisture in the heater, for example the electrolyte solution or any solution used to activate an electrolytic salt. In this sense, a waterproof air diffuser can be a two-way moisture barrier which prevents the entry of water or moisture into the interior 18 of the package while also preventing the escape of moisture trapped within the heater which is required to trigger the reaction once the heater substrate is exposed to air/oxygen. Such may prevent evaporation of electrolyte solution from the inside of the heater prior to or during activation, extending the storage and operational life of device 10.

As seen in FIGS. 7-12, package 12 may be divided into multiple compartments or cavities 34, 36. Within one cavity, cavity 34 for example, heater element 22 including the reducing agent and electrolyte solution pay be positioned. Isolated within a different part of the housing in cavity 36, for example, may be the air delivered composition integrated with second wicking layer 26 or formed as a substrate 27 within cavity 36. The cavities may be formed in package 12 by sealing front panel 14 and back panel 16 around each cavity and the elements provided therein. Each cavity may then be provided with air access openings 20. For example, air access openings 20 in either the front panel or back panel may be provided adjacent cavity 34 in order to ensure that air can enter the interior of cavity 34 to provide air and oxygen to heating element 22. Air access openings 20 may likewise be provided adjacent cavity 36 in order to ensure that any scent generated is emitted from the interior of the device for utilization.

Where multiple cavities are created, a single air diffuser 32 may be utilized for both cavities as seen in FIGS. 7-12. Likewise, a single removable seal 30 may be utilized to cover the air access openings adjacent each cavity. As seen in FIG. 12, where multiple cavities are provided, multiple removable seals 30, 31 may be utilized so that each cavity has a separate removable seal covering its associated air access openings 28. It is also contemplated that each cavity may be provided with its own air diffuser rather than using a single common air diffuser across both cavities.

When heating element 22 and the air delivered composition are separated into separate cavities within package 12, in order to insure heat transfer from the heating element to the air delivered composition in order to activate the air delivered composition, package 12 and front and back panels 14, 16, as well as air diffuser 32 should be made of a flexible material. By making the front and back panels and the air diffuser from a flexible material, the package can be folded along an axis or dimension 38 of the package as seen in FIGS. 11 and 12, allowing cavities 34, 36 to be brought into thermal contact with each other. By bringing the cavities into thermal contact with each other, the air delivered composition can be activated by the heater element when the heater element is activated and supplied with oxygen.

As a further alternative, as seen in FIGS. 13 and 14, with FIG. 14 being an exploded view of the device shown in FIG. 13, the air delivered composition, shown on a separate wicking layer 26 or formed as a substrate, may be housed in a cavity 39 formed integrated with or formed on an exterior side of front panel 14 or back panel 16 with heater element 22 being housed within interior 18 of package 12. In such embodiments, heat generated by the reducing agent when exposed to oxygen will be transmitted to cavity 39 causing the air delivered composition to activate. Where a separate cavity 39 is utilized, cavity 39 may be provided with air access holes 29 in any film or panel which forms the cavity, and a second removable seal 31 which may be placed over the air access holes. By providing cavities and a removable seal, releases or emissions by the air delivered composition may be more easily recognized outside the package. Cavity 39 may also include an air diffuser, waterproof otherwise between the air delivered composition formed as a wicking layer or substrate and an exterior film or panel bounding the cavity.

As previously mentioned, rather than form front and back panels 14, 16 from an air impermeable material, the front and back panels of package 12 may be formed using an air permeable material. Such and embodiment can be seen in FIGS. 15-17, with FIG. 16 being an exploded view of the package shown in the device of FIG. 15.

As seen in FIGS. 15-17, device 10′ includes package 12′ having front panel 14′ and back panel 16′ surrounding an interior 18′ which houses a heater 20′ which includes a heater element 22′ and a wicking layer 24′, as well as housing an air delivered composition in the form of a wicking layer 26′. Front and back panels 14′ and 16′ may be formed using materials similar to those discussed or used for air diffuser layer 32 in FIGS. 1-14. As such, front and back panels 14′, 16′ may be both air permeable and liquid impermeable or waterproof

When front and back panels 14′, 16′ are formed using air permeable materials, device 10′ may include a sachet or housing 40 which is formed with an air impermeable first panel 42 being sealed with an air impermeable second panel 44. Package 12′ may be sealed within sachet 40 in an air-tight manner in order to prevent air, and consequently oxygen, from reaching package 12′ inasmuch as any air which reaches package 12′ will reach interior 18′ and cause heater element 22′ to begin generating heat.

In order to reach package 12′ and expose heater element 22′ to oxygen to activate the air delivered composition, sachet 40 includes a removable portion 46. Once removable portion 46 is removed, air will reach interior 48 of sachet 40 where package 12′ is contained, causing heater element 22′ in package 12′ to begin generating heat, activating the air delivered composition therein. Package 12′ may be fixed within sachet 40 or may be partially or fully removable therefrom as seen in FIG. 17.

Sachet 40 may be resealable once removable portion 46 is removed. Resealing may be accomplished by re-engaging the removable portion 46 if removable portion 46 is an adhesive seal. Sachet 40 may also be resealable utilizing zipper locking wherein mating elements are placed on first panel 42 and second panel 44 to allow the sachet to be sealed shut after opening.

Whether device 10 or 10′ is utilized, it is contemplated by the invention that package 12 or 12′ may be integrated with a garment or patch which can be worn by a user to realize both the heat and aromatic effects of either device. As an example, either package 12 or 12′ may be integrated or inserted into a garment such as a facemask, wrist band, scarf, hat, glove, or other wearable element. An exemplary garment and patch configured to be integrated with package 12 or 12′ can be seen in FIGS. 18 and 19, respectively. Specialized garments or patches may be developed with receiving areas 50 for example, which are configured to receive an activated package 12 or 12′. Receiving areas 50 may be configured to provide insulation on any side which may contact a user, for example on a side of wristband which may contact a user's wrist. Lighter weight and/or highly permeable materials may be used on an exterior portion of receiving areas 50, for example, in order to maximize transmissivity of aromas from a package 12 or 12′ inserted into the receiving area.

Though the following improvements will be discussed with respect to various heaters, it should be understood that these improvements may be incorporated into any of the devices, packages or heaters of the preceding embodiments shown in FIGS. 1-17. Any of the improvements discussed herein with respect to the following heaters can be incorporated in any combination in substantially the same manner in devices 10, 10′, packages 12, 12′ and/or heaters 20, 20′ in the preceding embodiments.

As seen in FIGS. 20 and 21, with FIG. 21 being an exploded view of the heater shown in FIG. 20, heaters 100 include a housing or package 102 formed by a first film layer 104 and a second film layer 106. Disposed within interior 108 of housing 102 is heater element 110 which comprises a reducing agent and electrolyte solution as discussed herein. Rather than integrate the reducing agent and electrolyte into a single heater element, a wicking pad as discussed herein may be used to hold electrolyte and form a portion of heater element 110. The reducing agent and electrolyte may be formed as a single element integrated as a single substrate or with a pad as previously discussed, or as separate elements placed adjacent and in direct contact with each other to facilitate the transfer of electrolyte to the reducing agent. Housing 102 includes a removable portion 112 which may be removed to allow oxygen to reach interior 108 and heating element 110. An air diffuser 114 may be provided between one of the first film layer and the second film layer and the heater element.

In order to control the chemical reaction, the microstructure of the heater element, and in particular a heater substrate when used, may be specifically selected and designed to achieve specific porosities—both dry porosity before any “liquid” element is applied directly or indirectly to the heater substrate, and wet porosity which accounts for the amount of “liquid” applied directly or indirectly to the heater element and the aforementioned microstructure. According to the present invention, it is preferable that the wet porosity be in the range of 10%-90% after any “liquid” elements are applied either directly or indirectly to the substrate during production, and more preferably if in the range of 10%-90%, and more preferably in the range of 35%-80%, and more preferably yet in the range of 50%-80%.

The terms dry and wet porosity used herein refer to the porosity of the heater element. The dry porosity is determined by calculating the theoretical density of the components in the dry heater element and then subtracting the apparent density from the theoretical density. The difference is then divided by the theoretical density to determine the dry porosity. The wet porosity is determined by calculating the free volume in the dry heater element, subtracting the volume of activator solution added to the sheet to determine the final free volume and then dividing by the initial free volume and multiplying the complete result by the dry porosity. The wet porosity is essentially a reduction in the porosity of the substrate prior to the introduction of “liquid” elements, i.e. the dry porosity, as a result of the addition of “liquid” elements to the heater sheet directly or indirectly. “Liquid” elements include but are not limited to electrolyte solution and/or any liquid or oil air delivered compositions which are applied directly to the heater element or absorbed into the heater element from a saturated pad or the like which is placed adjacent the heater element during production.

Applicant has additionally found that by configuring the components of the heater element in a specific manner, for example by controlling the thickness and/or surface area and/or density of the heater element, as well as the microstructure of the heater element including the arrangement and internal structure of the heater element, wet porosities previously thought to be too high and lacking in electrolyte to sustain a reaction can be realized.

By controlling the weight of the reactant or reduction agent in the heater substrate, and the thickness, surface area and density of the heater substrate, porosities over 60% can be realized while the maximum temperature and heating time to the maximum temperature are controlled. For example, as the density (free volume) of the substrate increases, the ratio of liquid elements, i.e. electrolyte or liquid or oil air delivered composition, which can be added to the heater can be increased. An example of free volume within a heater element can be seen in FIG. 23 which shows a generic heater element 200 including a blow up of portion C which may be utilized as heater element 22, 22′ or 110 in any of the embodiments discussed herein. Free volume 202 existing within heater element 200 provide free volume which enhances porosity and provides area for any “liquid” elements to be absorbed into heater element 200.

Furthermore, as the weight of the reducing agent increases, the maximum temperature of the heater increases, as the additional reducing agent and additionally stored electrolyte (as a result of the increased density/free volume). However, with the increased density/free volume comes an increase in the amount of time in which it takes the heater to reach its maximum temperature.

By controlling the density and free volume of the heater element—specifically by increasing and decreasing the weight and amount of reducing agent—and thereby controlling the total amount liquid elements and the wet porosity of heater, heaters having specifically designed maximum temperatures and/or release times can be realized. For example, a heater having a small weight—and therefore a lower density/free volume and less reactant, which therefore means less electrolyte and/or liquid or oil air delivered compositions when added, and a higher wet porosity may be used for applications where a very fast, but perhaps lower temperature is required. The fast reaction due to the high wet porosity and lower temperature being due to less reducing agent and electrolyte solution to react with supplied oxygen and generate heat. By contrast FIG. 22 also shows the increased maximum temperature time and increased maximum temperature resulting from higher weight and denser heaters having higher free volume, and consequently more reactant and more electrolyte solution. Such heaters do however have an increased heating time specifically as a result of the lower wet porosity.

When a heater element is used in conjunction with a scented element, when an extended or delayed air deliverable release is desired, a heater having a longer heating time can be utilized with the air delivered composition being selected to deliver its deliverable at a particular temperature the heater will reach after a desired amount of time. When quick air deliverable release is required, a heater having a higher wet porosity can be utilized with an air delivered composition being selected which releases its deliverable at a lower temperature.

In order to more efficiently and better retain moisture within any of heaters 20, 20′, 100 or packages 12, 12′, 102, any of the heaters or packages of the present invention may further include materials which regulate moisture within the heater using moisture absorbing or complexing properties. As an example, Vermiculite is a material that reversibly retains or absorbs moisture and releases it as needed. In addition to Vermiculite, other metal silicates or other water complexing agents could also be used to provide and maintain a desired level of moisture within a heater. Other materials which may be incorporated into heaters 20, 20′ or 100 or packages 12, 12′, 102 that are contemplated as being used for moisture regulation, include but are not limited to, metal silicate complexes, reversible desiccants, molecular sieves, silica and polyacrylic acid salts. Absorptive minerals and clay may also be used as a moisture absorber within a package and/or heater. These materials may be included within the heater element itself or housed within any of packages 12, 12′, 102 in order to retain electrolyte solution or solution used to activate and electrolyte salt.

The above moisture absorbing materials may also or alternatively be used to replace a portion of reducing agent within a heater element, thereby reducing costs and improving efficiency with respect to the reducing agent used within the heater element. For example, in a typical heater as much as 70% of the reducing agent remains unused when the heater life has expired, typically as a result of the electrolyte solution being used up and/or evaporated from the heater. Using moisture absorbing materials, inert fillers or combinations of both may enhance the moisture retention within the heater element, and therefore electrolyte solution retention within the heater, while at the same time reducing the amount of reducing agent in the substrate. Such may not only reduce costs and make for more efficient heaters, but may also improve manufacture of the heaters insofar as the addition of a moisture absorption material, for example, may lead to better and more efficient absorption of electrolyte into a heater element during the packaging and manufacturing process, avoiding any leakage or loss of electrolyte which may inhibit heater operability. Increased electrolyte absorption and use of moisture absorbing filler in is particularly beneficial in heaters which are designed to have a higher weight, amount of reducing agent, density and free volume, and therefore an increased amount of electrolyte solution, as the increased amount of electrolyte solution can make packaging and manufacture of heaters much more difficult and can lead to higher evaporation amounts. By incorporating a moisture absorption agent into the heater element, electrolyte solution can be more efficiently and quickly absorbed into the heater element during manufacture and packaging, avoiding leakage and reduction in heater performance, as well as help prevent evaporation of electrolyte solution once the heater is packaged and subsequently activated.

Alternatively, it is contemplated that a moisture absorbing element may be formed as an additional substrate or element 116 within any of packages 12, 12′, 100, as seen for example in the heater in FIGS. 24 and 25, with FIG. 25 being an exploded view of the heater shown in FIG. 24.

In order to further control the heat release of any of heaters 20, 20′, 100, and consequently extend aromatic release of any incorporated air delivered composition, any of the heaters or packages 12, 12′, 102 may include a heat retention material which absorbs, stores and releases heat after the generating reaction of heat element 22, 22′, 110 is stopped.

An exemplary heat retention material which may be incorporated into any of heaters 20, 20′, 100 or packages 12, 12′, 100 is a phase change material. As the heater element is activated, any phase change material incorporated into the heater or package containing the heater will heat and begin to melt from a solid to a liquid, storing heat in its liquid form. Once the reducing agent is spent or oxygen is removed from the reducing agent, the liquefied phase change material will continue to emit heat at the melting temperature of the phase change material until the phase change material has complete a second phase change from the liquid phase back to the solid phase. Such a phase change material is discussed, for example, in U.S. Pat. Pub. No. 2014/0109889 to Applicant which is incorporated herein by reference. As discussed therein, a phase change material may be integrated with any of a heater element/substrate, may be formed on a separate substrate 118 in heater 100 as seen in FIGS. 26 and 27 with FIG. 27 being an exploded view of the heater shown in FIG. 26. Of course, substrate 118 may be integrated with any package or heater disclosed herein.

A further benefit of using a phase change material as a heat retention element is that the heat emitted by the heater and any associated deice can be limited. Furthermore, where an air delivered composition is incorporated into the device or heater, with enough air delivered composition, selecting a heat retention material which can maintain a temperature at which the air delivered composition in the heater is activated and delivered through the air after the heater element reaction has been stopped or the heater has expired. This allows for the deliverable to be released during both the life of the heater and during the release of heat from the heat retention element. Such allows for the use of smaller heaters with less reducing agent and electrolyte solution in heaters and devices which are utilized to release the deliverable through the air using a heat activated air delivered composition.

Further elements which may be utilized as heat retention materials include specific minerals, such as clay and other minerals, heavier or thicker mass that may include composites filled with particles having high specific heat capacities such as minerals, ceramics and glasses.

In order to further improve heat retention of any of heaters 20, 20′, 100 or packages 12, 12′, 102, or as an alternative to a heat retention material, a reflective material or layer 120 may be applied to the heater 20, 20′, 110 or package 12, 12′, 102, as seen in heater 100 in FIG. 28, for example. These reflective layers may be incorporated within any of the heaters or packages of the present invention in locations which maximize the heat retention. For example, a metalized film or thin foil layer may be positioned proximate the heater element, i.e. within radiant heat conduction of the heater element and reducing agent, to radiantly reflect heat back into the reducing agent. These layers may include but are not limited to tape layers on the inside of the top and/or bottom film as seen in FIG. 28, IR or radiant heat reflective film for the inside a panel forming the housing or packaging of a heater or package and/or other separate layers within the heater or package. When positioned in such a manner, film 120 may include any required apertures to match those of any adjacent air access openings formed in the package or housing. Other layers discussed herein may also incorporate IR reflectivity as appropriate in sections or integrally to help heat retention. Rather than be adhered as a tape, any metallized films or thin foil layers may be printed on an interior portion of any panels or films forming the housing or package of the device or heaters disclosed herein.

The shown in separate heaters and packages, it should be understood that any of the aforementioned layers may be combined in any combination to realize a heater or package containing one or more of the previous discussed elements or layers. Different elements may also be integrated with disposed in the heater or package in different manners. For example, a layer for a heat retention material may be used in a heater where a moisture absorbing agent is integrated with heater element 22, 22′ or 110. The manner in which each element is incorporated into the heater may be modified in order to realize a particular desired heater structure, heater performance or result.

As seen in FIGS. 29 and 30, with FIG. 30 being an exploded view of the heater shown in FIG. 29, it is contemplated by the invention that a heater may have an applicator or application device integrated with an outer portion thereof to provide a heated applicator. As shown in FIGS. 29 and 30, heater 300 includes a housing or package 302 which is formed with a first panel 304 and a second panel 306 surrounding an interior 308. Within interior 308 is a a heater element 312 which includes a reducing agent and electrolyte. As with all previous embodiments, the heater element may be formed, for example, as a heater sheet or heater substrate and may have electrolyte solution absorbed therein. Though not shown in FIG. 29 or 30, it should be understood that a separate wicking layer impregnated with electrolyte solution may also be used and positioned adjacent heater element 312 in order to deliver electrolyte solution to the heater element and the reducing agent. Heater 300 may also include an air diffuser 316 and a removable seal 318 covering air access openings 320.

Integrated with heater 300 is applicator 314 which is adhered or otherwise attached to an external portion of one of the first or second panels 304, 306 of housing or package 302. Applicator 314 may be a pad, sponge, woven or non-woven material or brush. Heater 300 may further include any of the aforementioned layers, materials or improvements discussed herein. For example, heater 300 may include an air delivered composition which is integrated with heater element 312 or provided on a separate layer or substrate within housing or package 302. It is contemplated that the air delivered composition may also be integrated with the applicator 314. Heater 300 may further include any combination of moisture absorbers, heat retention materials and/or reflective layers as discussed herein. Though shown attached to heater 300, it should be understood that applicator 314 may be attached to any of packages 12, 12′, sachet 40 or heaters 100 discussed herein in substantially the same manner as attached to heater 300.

FIGS. 31 and 32 show a further embodiment of the present invention wherein the device is an application device 400 having a substrate forms an application medium. A reducing agent which generates heat when activated and exposed to oxygen is integrated with the substrate, i.e. the application medium. An electrolyte solution is also integrated with the substrate, i.e. the application medium. As discussed in the previous embodiments, the electrolyte solution is an activator which causes the reducing agent to activate and generate heat when exposed to oxygen.

As seen in FIG. 31, the substrate or application medium may be solid element 402 like an applicator. Exemplary applicators which may have a reducing agent and electrolyte solution integrated therewith include but are not limited to a pad, sponge, non-woven or brush. In order to prevent activation of the heater prior to use, the substrate or application medium may be stored in an air tight container 404 prior to use. The removable of the substrate from the container 404 and exposing the substrate and stored reducing agent to oxygen will cause the substrate to heat.

As seen in FIG. 32, the substrate or medium may be a compound 406 which is liquid or semi-solid at room temperature. Compound 406 may be, for example, a lotion, cream or repellant and may be stored in an air tight container 408.

Whether solid, liquid or semi-solid, the substrate may further include an air delivered composition which is integrated with the substrate. The air delivered composition may generate an aroma, scent or repellant when heated by the reducing agent when the reducing agent is exposed to oxygen and activated to generate heat. It is contemplated that where the scent is a liquid or oil, such may also be transmitted from the substrate to a surface rubbed with the substrate when the substrate is solid.

It is to be understood that the aspects and objects of the present invention described above may be combinable and that other advantages and aspects of the present invention will become apparent to those having ordinary skill in the art upon reading the following description of the drawing and the detailed description thereof.

What is claimed is:

1. A device for the delivery of a deliverable through the air comprising:
a package having a front panel and a back panel;
a heater disposed inside the package between the front panel and the back panel; and
an air delivered composition, the air delivered composition being positioned to receive heat from the heater when the heater is activated, wherein the air delivered composition is volatilized by the heater when activated causing delivery of the air delivered composition to be delivered through the air.

2. The device of claim 1 wherein the heater comprises
a heater element comprising a reducing agent which generates heat in the presence of oxygen; and
an electrolyte solution, the electrolyte solution being an activator which causes the reducing agent to activate and generate heat when exposed to oxygen.

3. The device in claim 2, wherein the air delivered composition is integrated with the heater element.

4. The device in claim 2, wherein the air delivered composition is integrated with the electrolyte.

5. The device in claim 2, further comprising a pad, wherein the air delivered composition is integrated with the pad, the pad being disposed inside the package so as to receive heat from the heater when the heating element is exposed to oxygen and activated.

6. The device in claim 5, wherein the pad is disposed adjacent the heating element.

7. The device of claim 1, wherein the front panel and the back panel are both air impermeable, wherein at least one of the front panel or the back panel includes at least one air access opening, wherein the package further comprises at least one removable seal affixed to at least one of the front panel or the back panel covering the at least one air access opening.

8. The device of claim 7 wherein the package further comprises at least one air diffuser, the at least one air diffuser being disposed inside the package and positioned between the at least one air access opening and the heater element.

9. The device of claim 8 wherein at least a portion of the air delivered composition is integrated with the at least one air diffuser.

10. The device of claim 8, wherein the at least one air diffuser is waterproof

11. The device of claim 7, wherein the at least one removable seal is resealable over the at least one air access opening.

12. The device of claim 1, wherein the heater element is positioned in a first cavity of the package and the air delivered composition is positioned in a second cavity of the package, wherein the first cavity and the second cavity are formed as separate portions of the package, wherein the package is flexible and the heater element and the scent agent are separated within the package, the package being foldable along a dimension of the package so that the first cavity can be brought into thermal contact with a second cavity and heat generated by the heating element when activated can be thermally transmitted through the first cavity and the front panel or back panel of the package and the second cavity of the housing to activate the scent agent.

13. The device of claim 1, wherein the front panel and the back panel are both air permeable.

14. The device of claim 13 further comprising a sachet having a first panel and a second panel, both the first panel and the second panel being formed from a material which is air impermeable.

15. The device of claim 14, whereby the sachet includes one or more removable portions, wherein removal of the one or more removable portions exposes the package to air.

16. The device of claim 15 wherein the sachet is resealable after the one or more removable portions are removed.

17. The device of claim 13, wherein at least one of the front panel or the back panel are waterproof.

18. The device of claim 1, wherein the air delivered composition is positioned in a second cavity integrated with the package.

19. The device in claim 17, wherein the second cavity is attached to one of the front panel or the back panel of the package using adhesive.

20. The device of claim 1, further comprising a substrate, wherein one or more of the heater element, the electrolyte or the air delivered composition are printed on the substrate.

21. The device of claim 1, wherein the device is integrated into a garment.

22. The device of claim 1, wherein the device is integrated or inserted into a patch that is worn on the body.

23. The device of claim 1, further comprising an applicator, the applicator being integrated with an exterior surface of one of the first panel or second panel.

24-48. (canceled)

49. The device of claim 14, wherein the package is removable from the sachet.

50. The device of claim 14, wherein the package is fixed within the sachet.

51. The device of claim 21 wherein the garment is a facemask.

52-55. (canceled)
